(19) United States
(12) Patent Application Publication
(10) Pub. No.: US 2016/0074018 A1
(43) Pub. Date: Mar. 17, 2016

(54) Urine Specimen Collection Kit With Retractable/Adjustable Handle and Sponge

(71) Applicant: Michelle Marie Lependorf, Princeton, NJ (US)

(72) Inventor: Michelle Marie Lependorf, Princeton, NJ (US)

(21) Appl. No.: 14/948,739

(22) Filed: Nov. 23, 2015

(51) Int. Cl. A61B 10/00

(52) U.S. Cl. CPC A61B 10/007; A61B 2560/0406; A61B 2503/40; A61B 2503/06; A61B 2503/12; A61B 2503/08

(57) ABSTRACT

This invention is comprised of: (i) a clear, measured, tubular specimen collection container; and (ii) a thin retractable/adjustable handle that consists of a telescopic middle stem with attached twist-on cap at one end and an absorbent, sterile sponge at the other end. For specimen collection, the handle is fully extended to its maximum length and the sponge placed in the urine stream of a test subject for several seconds. The sponge is then inserted into the specimen collection container, which has a fixed, built-in sieve located in its lower half, with a center slit for urine dipstick testing. The saturated sponge is gently pressed against the sieve to collapse the retractable handle to its shortest length, while the cap is twisted close, sealing the specimen container. The action of twisting the cap to seal the container compresses the sponge against the sieve, causing urine to be expressed into the lower part of the specimen collection container.

### CROSS-REFERENCE TO RELATED APPLICATIONS

N/A

### STATEMENT REGARDING FEDERALLY SPONSORED RESEARCH

N/A

### REFERENCE TO SEQUENCE LISTING IN TABLE OR A COMPUTER PROGRAM LISTING COMPACT DISK APPENDIX

N/A

### BACKGROUND OF THE INVENTION

This invention started with my dog. My dog is a 16 month-old female Golden Retriever. She was recently diagnosed with and treated for a urinary tract infection. To insure that her prescribed course of antibiotics was successful, my veterinarian asked me to collect a urine sample for follow-up testing. Initially, I thought this would be an easy thing to do. Unfortunately, I learned that nothing could be further from the truth! After about 30 minutes of coaxing my dog to pee, she finally assumed the pee position, while I hastily moved to place a small Tupperware container in her urine stream. I managed to collect a bit of urine in the container, but the majority of it wound up on my hand, sleeve and arm! It was disgusting! It reminded me of the times I tried to get urine samples from my daughters when they were very little. While walking home with the dripping Tupperware container and my urine soaked extremities and clothing, I was beyond pissed off (no pun intended) and thought to myself, “There MUST be a better, less messy way to collect a urine specimen from dogs, children and people in general.” So, I designed this urine collection kit. It allows a person to more easily and with very little mess collect a urine sample from pets, children and other people in general, but especially those, for whom traditional urine specimen cup collection proves challenging.

### SUMMARY OF INVENTION

This invention is comprised of a clear, tubular specimen collection container and a thin retractable/adjustable handle that consists of a telescopic middle stem, a twist-on cap at one end and an absorbent, sterile sponge at the other end. The twist-on cap, telescopic middle stem and sponge comprise a single unit—referred to herein as the retractable/adjustable handle or simply the handle. To collect a urine specimen, the handle is first extended to its longest length and the sponge is thereafter placed in the urine stream of the test subject. Once collected, the saturated sponge is inserted into the collection container, which has a built-in, fixed sieve in its interior. The sieve has a tiny slit at its center to facilitate urine dipstick testing if desired. Following specimen collection at the source of urine output, the sponge is gently inserted into the specimen container and pressed against the sieve causing the retractable handle to be reduced to its shortest possible length, whereupon the cap may be twisted on tightly to seal the collection container. By twisting the cap to close the container, the sponge is compressed against the sieve causing the urine to be strained through the sieve and expressed into the area of the collection container beneath the sieve, where it is stored for later testing. The primary advantage of this invention is that it reduces the likelihood of getting urine on one's hands or clothing while attempting to collect a urine specimen. It also reduces the likelihood of sample contamination in that the sample goes directly from the source of output into the sealed collection container. In addition, because of the relatively small size of the handle and sponge, it is much easier to collect urine samples in tight spaces, such as the limited space afforded by a crouching, peeing dog, or from test subjects who have difficulty trying to urinate into traditional specimen cups, like small children or pregnant and elderly women. Traditional urine specimen cups often prove too large for the thigh widths of small children while seated on a toilet and hard to properly place by pregnant women and elderly women.

### DRAWINGS

The drawings attached to this application are contained on one page and fully demonstrate the invention.

FIG. 1 shows the retractable/adjustable handle with sponge at one end (Part A), middle telescopic stem (Part B) and twist-on cap at the other end (Part C).

FIG. 2 shows a clear, measured, specimen collection container (Part D). The collection container has a built-in sieve (Part E) and the sieve has a centered slit for urine dipstick testing if desired.

### DETAILED DESCRIPTION OF THE INVENTION

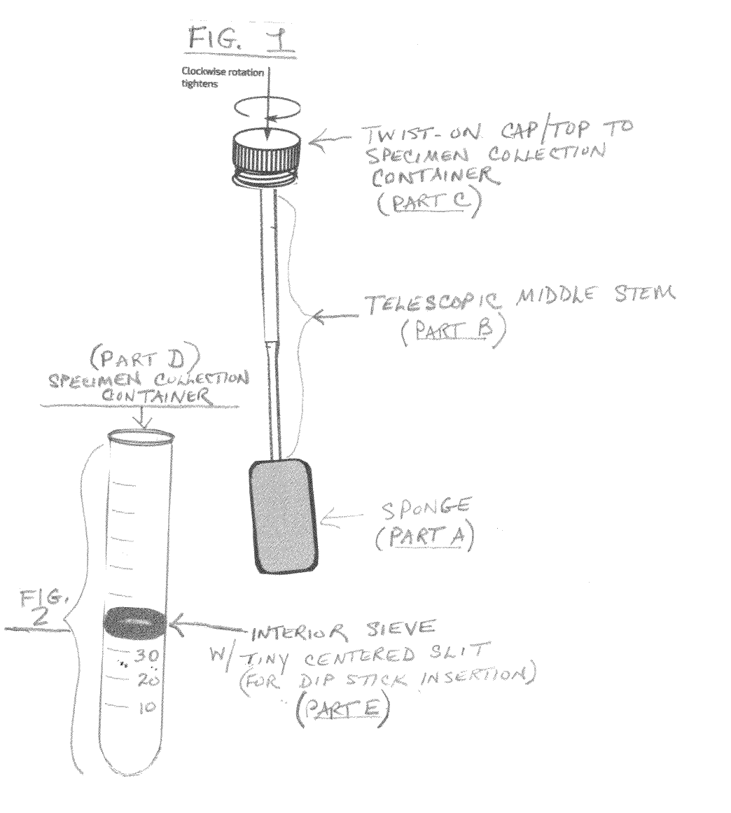

This invention is of very simple design, consisting of only two separate primary pieces—(1) a clear, tubular, urine specimen collection container with cc measurement markings and built-in sieve; and (2) a thin retractable/adjustable handle comprised of a telescopic middle stem, twist-on cap and sponge. (See Drawing, FIG. 1 and FIG. 2). The handle's telescopic middle stem is joined to a twist-on cap at the top of the handle, which cap is used to cover and seal the specimen container, while the lower portion of the middle telescopic stem is joined to a sterile, absorbent sponge. The sponge is used to absorb urine at the source of urine output, after which it is placed inside the specimen collection container. The collection container is a traditional tubular specimen collection container with graded/notched volume measurements extending from 5 cc to 30 cc. Just above the 30 cc mark there is a fitted, built-in, sieve (See Drawing, Part E) that has a small slit at its center to accommodate a urine dipstick test if desired. The invention is utilized in the following way: the retractable telescopic handle is first extended to its full length for specimen collection at the source of urine output. Next, the sponge is placed into the test subject's urine stream. Once the sponge has been held in the urine stream for several seconds, the sponge is inserted into the specimen collection container. The cap is then pushed down and the sponge is gently pressed against the sieve causing the retractable/adjustable handle to reduce or collapse to its smallest length. The cap is then twisted close, which will result in slight compression of the sponge against the sieve, thereby causing the collected urine to be strained or expressed through the sieve into the lower part of the specimen collection container. Once collected and secured in the container, the urine is stored there for later testing. With the exception of the sponge, all parts are made of disposable plastic.

1. A urine specimen collection kit that is comprised of: (a) a clear, tubular measured specimen collection container having a built-in sieve, which sieve is interiorly situated with a centered slit for urine dipstick collection; and (b) a thin retractable/adjustable handle that is comprised of a telescoping middle stem, with twist-on cap at one end and an absorbent, sterile sponge at the other end.

2. A urine specimen collection kit as described in claim 1 above that is capable of collecting urine from the source of urine output at a distance of at least 8 inches, which, in turn prevents the hands/arms and clothing of the specimen collector from coming into direct contact with urine at the point of urine output.

3. A urine specimen collection kit as described in claim 1 above that uses a sterile absorbent sponge to collect urine at the source of urine output, which sponge is then immediately inserted into the paired specimen collection container, where it is compressed by closure of the twist-on cap, thereby pressing the sponge against the built-in sieve causing the collected urine to be strained or expressed into the specimen collection container, a mechanism that avoids the necessity of directly collecting urine in a liquid form by hand using a traditional urine specimen cup, which can and often does slip from the collector's hand, spill and/or otherwise slosh around contaminating all surfaces with which it comes into contact.

4. A urine specimen collection kit as described in claims 1 thru 3 above that reduces the risk of urine sample contamination because of its ability to more readily collect a “clean catch” or sample of urine from the source of urine output, followed by its immediate placement and storage in a sealed specimen collection container.

5. A urine specimen collection kit as described in claims 1 thru 3 above that facilitates effective collection of urine samples in small, confined or tight spaces, such as when trying to collect urine samples from pets and small children, by use of the retractable/adjustable handle described in claim 1 above.

6. A urine specimen collection kit as described in claims 1 thru 3 above that facilitates the collection of urine samples from test subjects for whom collecting urine samples is often difficult or impossible using the traditional urine specimen cups due to both spatial and physiological limitations.

7. A urine specimen collection kit as described in claims 1 thru 3 above that permits easy urine dipstick testing through the tiny slit in the sieve described in claim 2 above, as well as traditional culture-based testing of the urine sample.

8. A urine specimen collection kit as described in claims 1 thru 3 above that can be utilized to collect urine samples from pets, children, pregnant women, elderly women and any other people who are unable to use or are challenged in the use of traditional urine specimen cups.
